(19) United States
(12) Patent Application Publication
(10) Pub. No.: US 2008/0312700 A1
(43) Pub. Date: Dec. 18, 2008

(54) Bioabsorbable Screw

(76) Inventor: Adam James, London (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/574,467

(22) PCT Filed: Jul. 12, 2005
(86) PCT No.: PCT/GB2005/002717

§ 371 Date: Feb. 28, 2007

(30) Foreign Application Priority Data
Aug. 28, 2004 (GB) 0419241.5

(51) Int. Cl. A61B 17/04

(52) U.S. Cl. 606/304; 606/301; 606/309

(57) ABSTRACT

The invention provides a bioabsorbable screw (1), suitable for use in orthopaedic surgery, and having cavities (8) disposed on the surface of the shaft. In particularly preferred embodiments of the invention, grating (or abrading) means are provided, and the screw shaft may be hollow, to assist eves biodegradation of the screw. The use of low melting point polymers (particularly caprolactone) further allows the screw to be melted in-vivo, producing increased fixation strength.

### FIELD OF THE INVENTION

The invention relates to bioabsorbable screws, and particularly those used for fixing orthopaedic implants to or in a bone of a patient.

### BACKGROUND AND PRIOR ART KNOWN TO THE APPLICANT

Many procedures in the field of orthopaedics require the use of screws to secure implants to or within a bone of a patient. One such use is in the reconstruction of the anterior cruciate ligament (ACL), which will be used in this specification to illustrate the invention. Metal screws (often made of titanium) are used for this purpose currently and are the preferred choice for surgeons as they cause minor inflammatory response. However, this often requires a second surgical intervention to remove the screw after healing. Also, because mechanical stresses are borne to a large part by rigid metal screws, the surrounding bone does not carry sufficient load during and after the healing process to produce a biologically strong structure. In some cases this can cause rise to post operative complications a number of years after implantation.

Synthetic biodegradable polymers are currently available and are an alternative choice to metal screws. As the polymer is degraded and absorbed by the body during the months following surgery, the screw site is replaced by biological tissue and so the biomechanical stresses are transferred from the implant or screw to the newly-formed tissue produced during the healing process. A typical application for this type of screw is in the reconstruction of the anterior cruciate ligament, which connects the tibia to the femur. It serves to prevent the tibia (shin) from moving forward relative to the femur (thigh). The ACL is in the centre of the knee and crosses the posterior cruciate ligament (PCL). It is frequently injured in contact sports (such as rugby) and pivoting sports (such as soccer and skiing)—although there are many scenarios for injury to this ligament. Usually the patient complains of a sudden injury to the knee and the inability to walk after the accident. Surgical reconstruction of the ACL is necessary to stabilise the femur in relation to the tibia.

Although a number of different types of tissue have been utilised to reconstruct the ACL, the most common technique involves harvesting the central third of the patellar tendon with a bone block at each end of the tendon graft. This is only harvested after performing a diagnostic arthroscopic examination of the knee. The remaining patellar tendon is then repaired. After harvesting the graft, drill guides are used to make 7-10 mm holes in the tibia and femur. By placing the drill holes at the sight where the original ACL would have attached to the bones, the graft, when applied, will fulfil the same function and provide the comparable stability to the original ACL. After pulling the graft through the drill holes and into the joint, it is then secured in place with bioabsorbable or metallic screws. The screws used in this procedure are solid, generally cylindrical in shape, with an external thread and may be made of a bioabsorbable polymer. A typical example would be the bioabsorbable screws sold under the trade name BioRCI by Smith and Nephew Inc, Andover, Mass., USA. In the technique employed, the harvested tendon is laid within the drilled hole and the screw is inserted alongside each bone block to provide an interference fit within the tunnel. To avoid damage to the tendon, the threads are constructed of a soft polymer to protect the tendon graft.

A problem with the current methodology and associated fixings is that the trauma caused by the size of the tunnels brings about a number of post-operative complications that affect the integrity of the fixation. The tunnels get filled with synovial fluid and rarely re-ossify with new bone. The tunnel is eventually filled with a fibrous mass, which presents itself as a weak point for future complications. The size of the tunnels also reduce the biological healing rate which results in prolonging the mobilisation of the joint vital to stimulate blood flow and the biological processes to nourish and strengthen the graft. All of these factors result in a less than optimal final strength of the reconstructed ACL. It is among the objects of the present invention to attempt a solution to this problem.

### SUMMARY OF THE INVENTION

In its broadest aspect, the invention provides a bioabsorbable screw, having a shaft and a thread, said thread having a crest and a root region, characterised by the provision of one or more cavities in the exterior face of the shaft and located in the root region of the thread. Preferably, the bioabsorbable screw is further characterised by having a hollow shaft, and wherein one or more cavities communicates with the hollow interior of the shaft.

Preferably, and in any aspect of the invention, the bioabsorbable screw is further characterised by the provision of grating means located in the root region of the thread. More preferably, the grating means is formed as part of the lip of a cavity.

When the bioabsorbable screw is hollow, it is preferably further characterised by being of a multi-part construction, so allowing the screw to be split apart to give access to the hollow interior of the shaft. More preferably also, the bioabsorbable screw further comprises a recess, in communication with the hollow interior, so as to anchor, in use, an orthopaedic implant, or a graft.

In any aspect of the invention, the bioabsorbable screw is preferably made substantially of a material with a melting point below 70 degrees Celsius. Most preferably, the screw is made of polycaprolactone (PCL).

The invention also provides a method of fixing an orthopaedic implant or graft to or in a bone of a patient, comprising the steps of: forming a guide hole in the bone of a patient; fixing an orthopaedic implant using a biodegradable screw with a low melting point; and melting at least part of the screw. Preferably, the melting is carried out by the use of power ultrasound.

### BRIEF DESCRIPTION OF THE DRAWINGS

The invention will be described by reference to the accompanying drawings in which:

FIG. 1 is a schematic cross-section of a screw and illustrating its various features;

FIG. 2 is a cross-section of part of a screw of the present invention illustrating surface cavities;

FIG. 3 is a schematic cross-section of part of a screw of the present invention illustrating abrasive/grating elements;

FIG. 4 is a schematic illustration of a cross-section of part of a screw of the present invention illustrating a hollow interior;

FIG. 5 is a schematic illustration of a cross-section of part of a screw according to the present invention illustrating a hollow interior and abrading/grating elements;

FIG. 6 is a cross-section through a grating element formed as part of the lip of a cavity;

FIG. 7 is a cross-sectional view of a screw according to the present invention illustrating, in particular, a recess forming an anchor position;

FIG. 8 is a schematic elevation of a screw according to the present invention;

FIG. 9 is a schematic perspective view of a screw according to the present invention; and

FIG. 10 is an exploded perspective view of a screw according to the present invention; and

FIG. 11 is an end elevation of a screw according to the present invention.

### DESCRIPTION OF THE PREFERRED EMBODIMENTS

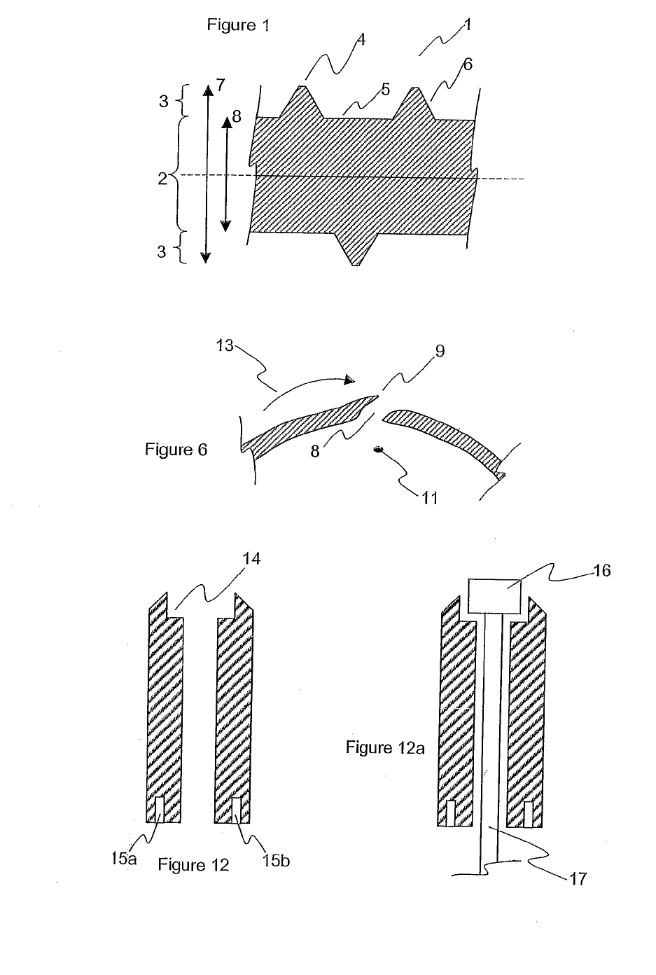

FIG. 1 is a generalised cross-section through a threaded screw (generally indicated by 1) to illustrate the terminology employed in this description. The thread comprises a shaft portion 2 and a thread portion 3. The thread 3 has a crest 4 (i.e. the prominent part of the thread) and a root 5 (i.e. corresponding to the shaft between adjacent crests). The flanks 6 of the thread are the sides that connect the crest and the root regions. These parts of the thread define a major diameter indicated by the arrow 7, i.e. the diameter that just touches the crest of the external thread and a minor diameter 8 that just touches the root of the thread.

In one embodiment of the invention, there is provided a bioabsorbable screw as illustrated in the partial cross-section of FIG. 2. In this embodiment, the screw has one or more cavities 8 in the exterior base of the shaft 2 of the screw and located in the root region 5 of the thread. As the screw is driven into a pre-drilled hole within a bone, small flakes of bone and tissue will be released as the thread cuts into the bone. The resulting ‘bone mulch’ will be deposited within the cavities 8 in the shaft of the screw. In this way, re-ossification of the bone, and biological healing around the graft stump in the tunnel will be enhanced.

FIG. 3 illustrates an alternative embodiment of the invention whereby grating means (9, 10) are provided in the root region 5 of the thread. The grating means may comprise abrasive protrusions 10, most preferably located between the minor and major diameters of the screw, or may be in the form of a cutting element. A particularly preferable means of providing such a cutting surface is by forming it into the lip of a cavity as illustrated in the cross-sectional view of FIG. 6 (to be described in more detail below).

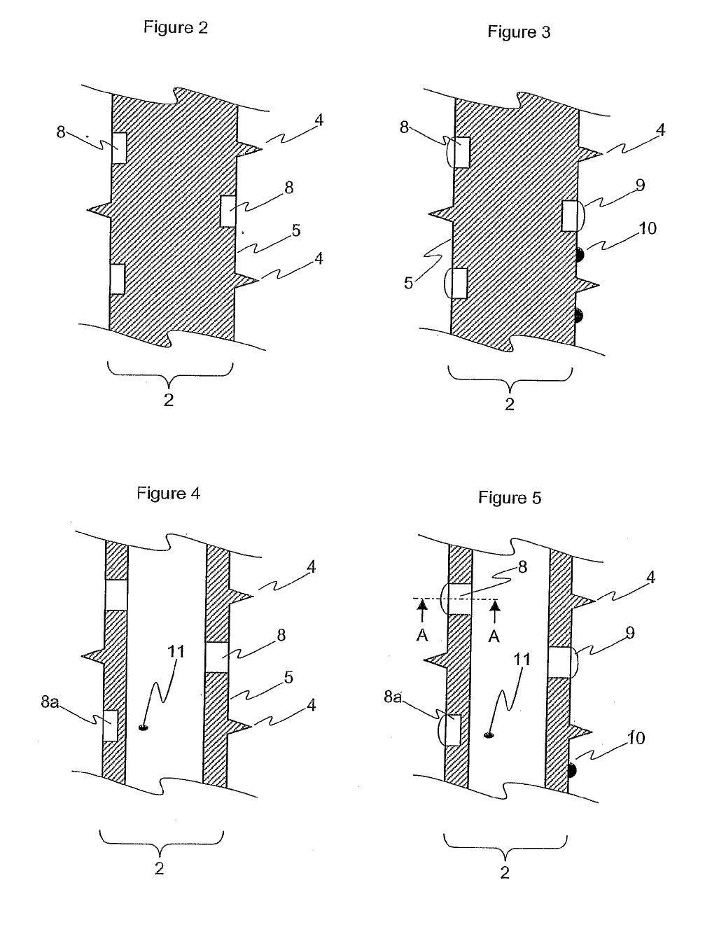

In a further, and more preferable, embodiment of the invention, illustrated in FIG. 4, the shaft 2 of the screw is hollow. In this embodiment, one or more of the cavities 8 communicates with the hollow interior 11 of the shaft 2. Other cavities (e.g. 8a) may solely be surface features. In this embodiment, the bone mulch is able to migrate through the cavities 8 of the screw and into the hollow interior 11 of the shaft 2. Bone mulch can also be harvested by the operating surgeon, pre-filling this cavity with bone mulch prior to insertion.

In the most preferred embodiment of the invention (illustrated in FIG. 5) the shaft 2 of the screw again has a hollow interior 11 and cavities 8 in communication with the hollow interior 11. Grating means are also provided within the root 5 of the thread, either as abrasive protrusions 10 or as a cutting element 9. In one particularly preferred embodiment, the grating means is formed as part of the lip of a cavity 8.

FIG. 6 illustrates a detail of one particularly preferred embodiment, in which the grating means is formed as part of the lip of a cavity 8. The figure is a partial cross-section (A-A) of a screw, illustrated in FIG. 5. In this embodiment the grating means 9 is formed as part of the lip of a cavity 8, and is raised above the surface of the screw shaft. In this way, as the screw is rotated in the direction indicated by the arrow 13, chips of bone and other tissue will be actively removed by the grating means 9 and directed through the cavity 8 and into the interior of the screw 11. Thus, the grating means 9 has the form typically associated with a cheese grater (when the cavity 8 extends into the interior 11 of the screw) or the form of a rasp (when a cavity, such as 8a in FIG. 5, is merely a surface feature).

FIG. 7 illustrates a cross-section through a highly preferred embodiment of the invention wherein a recess 14 is provided, in communication with the interior 11 of the screw, to anchor, in use, an implant. This feature is further illustrated in FIGS. 12 and 12a. This shows a cross-section through the screw and the recess 14. FIG. 12a illustrates how, in use, and for anterior cruciate repair, a bone block 16 and attached tendons 17 would be located in the recess 14. Also provided in this embodiment, are two or more recesses 15 in the end of the screw, these allow the screw to be turned by insertion of a prong-shaped tool allowing turning of the screw without damage to the implant (16, 17).

These slots are illustrated also in FIG. 11, which is an end view of the screw. In this embodiment, two such slots (15a, 15b) are provided on either side of the hollow interior 11 of the screw. In this particular embodiment, the screw is of two-part construction and may be split apart along the line 18 to allow access to the interior 11 of the screw.

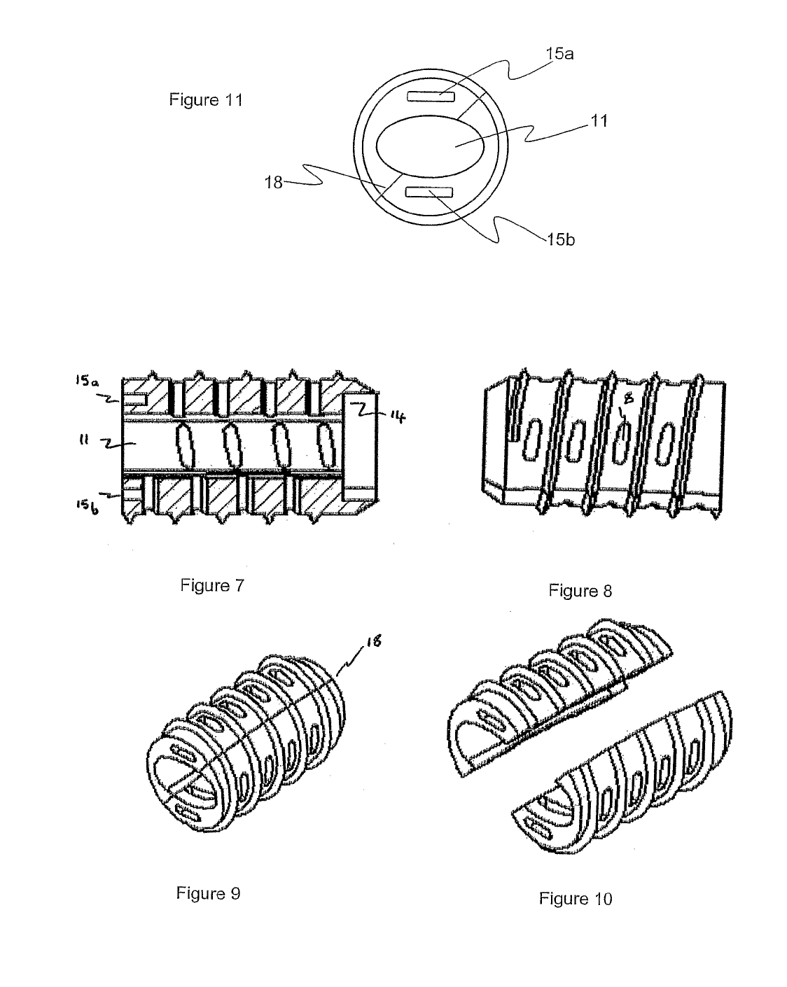

FIGS. 8, 9 and 10 show an elevation, perspective and exploded perspective view respectively of this highly preferred embodiment.

In any of the embodiments described above, the screw is made of a bioabsorbable material. Although suitable synthetic biodegradable polymers will be known to those skilled in the art, the skilled addressee is directed towards a recent review of these materials (Middleton, J. C., Tipton, A. J., ‘Synthetic Biodegradable Polymers as Medical Devices’, Medical Plastics and Biomaterials. 1998, 5 (2), 30-39) where a number of suitable candidates are described.

It is an object of this present invention to encourage a more even and rapid biodegradation of the screw implant, and to this end, the use of a polymeric material with a low melting point (below approximately 60° C.) has particular advantages. By causing the bioabsorbable screw to melt during or after insertion into a bone, the bone chippings and other biological material that have be placed or scraped into surface cavities 8, or into the interior 11 of a screw mix with the bioabsorbable polymer. By choosing a polymer with such a low melting point, it is possible to achieve this melting and mixing with biological material without damage to the latter. Amongst the candidate low melting point polymers, polycaprolactone is particularly advantageous, as it possess not only a low melting point (which is controllable by manipulation of the molecular weight) but has particularly suitable mechanical and biocompatibility properties. The polymer is also particularly good for loading further biological and pharmaceutical agents, for example: hydroxyapatite and calcium

The invention also, therefore provides a new method of fixing an orthopaedic implant to or in a bone of a patient, comprising the steps of forming a guide hole in the bone of a patient, for example by drilling; fixing an orthopaedic implant—such as a harvested bone-tendon-bone implant in the case of anterior cruciate ligament repair, and melting at least part of the screw. The melting process may be carried out during the insertion of the screw, either continuously or at intervals, or may be performed after the screw has been fully located. In this way, two particular benefits are achieved: firstly, the melting process causes the polymer and the bone mulch to mix dispersing the biological actives within the matrix of the material, enabling a more even degradation of the bioabsorbable screw; secondly, the melted polymer also migrates into the cancellous cavities of the bone, leading to highly increased fixation strength.

In a particularly preferred embodiment of this technique, the melting may be carried out by the use of power ultrasound.

1. A bioabsorbable screw, having a shaft and a thread, said thread having a crest and a root region, said shaft having an exterior surface provided with one or more cavities located in the root region of the thread; and wherein grating means are located in the root region of the thread.

2. A bioabsorbable screw according to claim 1, wherein the shaft has a hollow interior, and wherein one or more cavities communicate with the hollow interior of the shaft.

3. A bioabsorbable screw according to claim 1, wherein said one or more cavities has a lip forming the grating means.

4. A bioabsorbable screw according to claim 2, having a multi-part construction, so allowing the screw to be split apart to give access to the hollow interior of the shaft.

5. A bioabsorbable screw according to claim 2, further comprising a recess in communication with the hollow interior, so as to anchor, in use, an implant.

6. A bio absorbable screw according to claim 1, made substantially of a material with a melting point below 70 degrees Celsius.

7. A bioabsorbable screw according to claim 6, wherein the screw is made of polycaprolactone.

8. (canceled)
